(19) United States
(12) Patent Application Publication
(10) Pub. No.: US 2013/0338603 A1
(43) Pub. Date: Dec. 19, 2013

(54) CLOSURE FOR A SYRINGE AND METHOD OF PRODUCING SAME

(71) Applicants: Tilman Roedle, Wolfegg (DE); Paul Nelles, Ravensburg/Bavendorf (DE)

(72) Inventors: Tilman Roedle, Wolfegg (DE); Paul Nelles, Ravensburg/Bavendorf (DE)

(73) Assignee: VETTER PHARMA-FERTIGUNG GMBH & CO. KG, Ravensburg (DE)

(21) Appl. No.: 14/002,507

(22) PCT Filed: Feb. 23, 2012
(86) PCT No.: PCT/EP2012/000785

§ 371 Date: Aug. 30, 2013

(30) Foreign Application Priority Data
Mar. 3, 2011 (DE) 10 2011 013 791.2
Jul. 15, 2011 (DE) 10 2011 107 276.8
Aug. 24, 2011 (DE) 10 2011 111 552.1

(51) Int. Cl. A61M 5/31; B29D 99/00

(52) U.S. Cl. CPC A61M 5/3134; B29D 99/0096
USPC 604/199; 264/250

(57) ABSTRACT

A syringe has a syringe cylinder and a needle attachment piece provided at a distal end thereof of the syringe cylinder. The syringe has a closure including a closure cap that closes off the needle attachment piece, forming a seal, and a securing cap. The securing cap surrounds the closure cap and is attached to the needle attachment piece by way of a holding ring. At least the securing cap and the closure cap are configured in one piece.

The invention relates to a syringe according to the preamble of claim 1, a closure for a syringe according to the preamble of claim 4, and a method for the production of a closure according to the preamble of claim 11.

Syringes, closures and methods of the type discussed here are known. Known syringes have a syringe cylinder and a needle attachment piece provided at the distal end of the cylinder. In order to tightly seal such a syringe, which is preferably pre-filled with a medical and/or pharmaceutical composition, during its storage or before its use, it has a closure. This closure comprises a closure cap that closes off the needle attachment piece, forming a seal, as well as a securing cap that surrounds the closure cap and is attached to the needle attachment piece by way of a holding ring. The syringe, which is preferably suitable and intended for medical purposes, is therefore tightly sealed in the region of the needle attachment piece during its storage, by means of the closure cap, so that a preparation provided in the syringe cannot escape and cannot be contaminated by substances and/or germs that penetrate from the outside. Known closures are composed of separate parts. In particular, the securing cap and the closure cap are provided as separate parts. They are produced in separate production steps and joined together for assembly in the syringe. Because these are small parts, they can easily be lost. The separate production steps and separate storage of the individual parts leads to significant logistics effort. Because of the additionally required assembly step for joining the separate parts of the closure together, production of the closure and thereby also of the syringe becomes more expensive.

It is therefore the task of the invention to create a syringe, a closure and a method for the production of the closure, which do not demonstrate the aforementioned disadvantages.

This task is accomplished in that a closure having the characteristics of claim 1 is created. This closure is characterized in that at least the securing cap and the closure cap are configured in one piece. They can therefore be handled and stored together, so that one of the two parts cannot get lost; they also do not have to be joined together before or during final assembly of the syringe. As a result, the logistics effort is significantly reduced and an additional production step is eliminated.

Further advantageous embodiments are evident from the dependent claims.

The task is also accomplished in that a closure for a syringe having the characteristics of claim 4 is created. This closure has a closure cap for tightly sealing the syringe, a securing cap that surrounds the closure cap, a holding ring for fastening the closure onto the syringe, and a securing ring that surrounds the holding ring, on which the securing cap is removably attached. The closure is characterized in that at least the closure cap and the securing cap are configured in one piece. In this connection, the advantages already addressed in connection with the syringe are obtained.

A closure is preferred that is characterized in that the securing cap, the holding ring and/or the securing ring comprises/comprise a comparatively hard material. In this way, the securing cap can support the closure cap and, in particular, absorb forces that would otherwise lead to unintentional removal of the closure cap. The holding ring and the securing ring demonstrate the required stability for fulfilling their holding and securing function, respectively, if they comprise a hard material. Particularly preferably, the hard material is a thermoplastic polymer, very particularly preferably polypropylene. It is also preferred that the securing cap, the holding ring and/or the securing ring consists/consist of the hard material, preferably the thermoplastic polymer, very particularly preferably polypropylene.

A closure is also preferred that is characterized in that the closure cap comprises a soft or elastic material. This allows elastic and therefore close contact of the closure cap against the needle attachment piece, which preferably has a greater hardness than the closure cap. The soft or elastic material is preferably a thermoplastic elastomer (TPE). Particularly preferably, the closure cap consists of the soft material, preferably of TPE.

A closure in which the securing cap and the closure cap are produced in a two-component injection-molding process, as a one-piece element, is also particularly preferred. In this connection, it is possible to form the securing cap from a comparatively hard material, and the closure cap from a comparatively soft or elastic material, where the two are produced in one piece and in one and the same process.

Further advantageous embodiments are evident from the dependent claims.

The task is also accomplished in that a method for the production of a closure having the characteristics of claim 11 is created. This method is characterized by the following steps: A securing cap is injection-molded from a first, preferably hard material. A closure cap is injection-molded from a second, preferably soft material. In this connection, either the closure cap is injection-molded onto the securing cap, or the securing cap is injection-molded onto the closure cap. Ultimately, it therefore does not matter in what sequence the parts are formed by means of injection-molding. The essential thing is that the securing cap and the closure cap are produced in one piece from different materials, where the one element is formed onto the other element during the injection-molding process.

A method is preferred in which the securing ring is formed in the same multi-component injection-molding process as the securing cap and the closure cap. Preferably, the securing ring can be formed onto the securing cap. Particularly preferably, the holding ring is also formed in the same multi-component injection-molding process. In this way, method steps that must otherwise be carried out separately can be integrated into the one multi-component injection-molding process.

A method in which the securing cap, the securing ring, and preferably also the holding ring are formed in one step, from the same material, is particularly preferred. In this case, it is particularly possible that the entire closure is produced as a one-piece element, using the multi-component injection-molding process.

Finally, a method is preferred in which the securing cap and the closure cap are locked into one another during injection molding. For use of the syringe, the securing cap is removed, with the closure cap being carried along by the securing cap when the former is locked into the latter. The needle attachment piece is then accessible in one step, and the securing cap and the closure cap do not need to be removed separately.

Further preferred embodiments are evident from the dependent claims.

The invention will be explained in greater detail below, using the drawing. This shows:

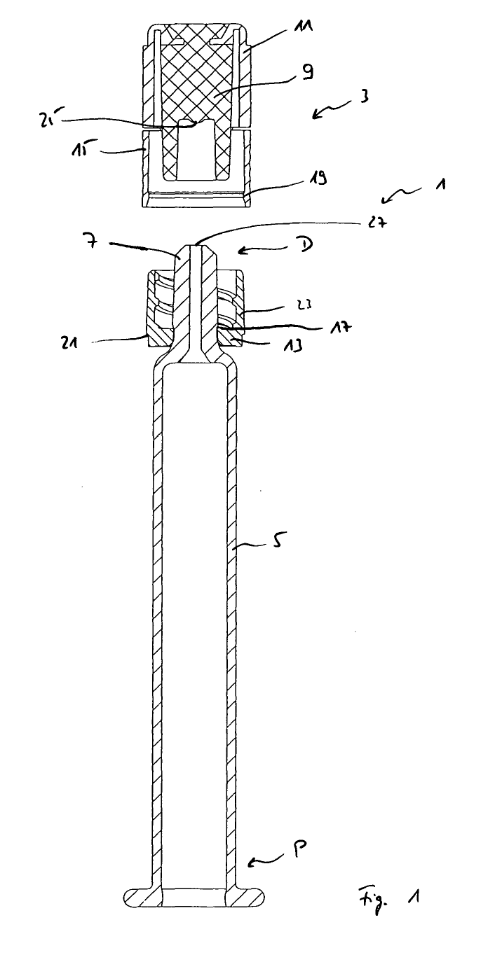

FIG. 1 a sectional representation of an exemplary embodiment of a syringe, with the closure not yet completely set on, and

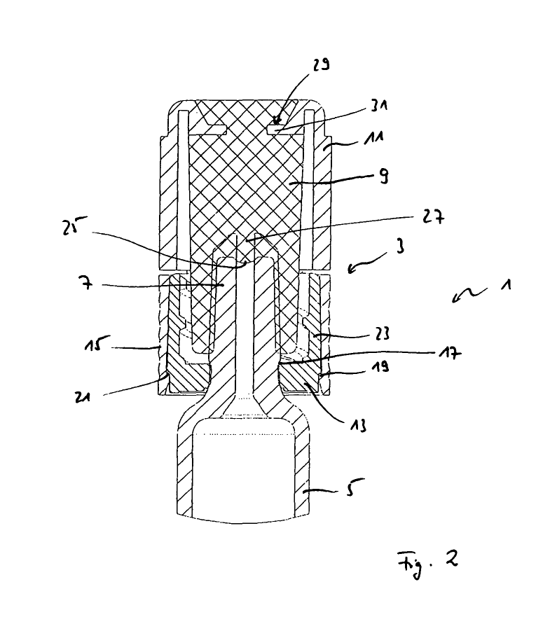

FIG. 2 a detail view of the sectional representation of a syringe in the closed state.

FIG. 1 shows a representation of an exemplary embodiment of a syringe 1 in longitudinal section, in a state in which a closure 3 has not yet been completely set onto the syringe 1. The syringe 1 has a syringe cylinder 5 that preferably accommodates a medical and/or pharmaceutical preparation. The syringe 1 preferably serves for medical purposes. The syringe cylinder 5 of the preferably pre-filled syringe 1 is preferably closed off, in the region of a proximal end P, by means of a plug, not shown. The plug can be displaced in the interior of the syringe cylinder 5, in order to eject the formulation by way of a needle attachment piece 7 provided at a distal end D.

This piece serves for coupling the syringe 1 with a dispensing means. As the term needle attachment piece indicates, this can be a needle or cannula. However, it is also possible to couple other dispensing means that are usual in the medical sector with the needle attachment piece 7, for example a tube of an injection system or of another medical device.

The closure 3 comprises a closure cap 9, which is configured in such a manner that the needle attachment piece 7 can be closed off with it, forming a seal. The closure furthermore comprises a securing cap 11 that surrounds the closure cap 9.

In the completely set-on state, the securing cap 11 is attached to the needle attachment piece by way of a holding ring 13. For this purpose, a securing ring 15 is preferably provided, on which the securing cap 11 is removably attached. Particularly preferably, at least one tear-off strip that connects the securing cap 11 and the securing ring 15 is provided, so that these elements are more or less connected with one another by way of a planned breaking point. In an exemplary embodiment, it is possible that multiple tear-off strips—preferably distributed at equal angle intervals from one another in the circumference direction—are provided, which connect the securing ring 15 and the securing cap 11 with one another.

It is also possible that projections are provided on the securing ring 15 and/or the securing cap 11, which projections extend in the direction toward the other element. Support forces can be introduced into these projections when displacement of the securing cap 11 and of the securing ring 15—in the longitudinal direction of the syringe 1, in other words in the axial direction—toward one another takes place. Thus, it is avoided that the tear-off strips tear unintentionally when axial pressure forces are introduced into the securing cap 11. These strips should, however, tear as intended when axial tension forces are introduced into the securing cap 11.

Preferably, the syringe 1 has a fastening means in the region of the needle attachment piece 7, preferably a groove, a bead or—as in the exemplary embodiment shown here—an undercut 17, by means of which the holding ring 13 can be fastened onto the needle attachment piece 7. In the exemplary embodiment shown in FIG. 1, the ring is pushed onto the needle attachment piece 7 from above and engages below the undercut 17. Then, it is preferably no longer possible to pull it off again without destroying it.

The securing ring 15 has a preferably ring-shaped projection 19, which projects radially inward, with which it engages behind an undercut 21 on the holding ring 13 in the set-on state. In the exemplary embodiment shown in FIG. 1, it is therefore pushed onto the holding ring 13, preferably also from above, and finally engages behind the undercut 21 with the projection 19. It is then preferably no longer possible to separate the securing ring 15 from the holding ring 13 in destruction-free manner. For this reason, the securing cap 11 tears off from the securing ring 15 in the region of the at least one tear-off strip when axial tensile forces are introduced into it.

The needle attachment piece 7 is preferably configured as a cone, very particularly preferably as a Luer cone. The holding ring 13 preferably comprises a wall region 23 that extends in the direction of the needle attachment piece 7, in other words in the axial direction, which region is configured, in the exemplary embodiment shown, as a thread, preferably as a Luer thread. In this way, the Luer Lock system, which is known and can be used in versatile manner, can be implemented on the syringe 1, as a whole.

The closure cap 9 and the securing cap 11 are configured in one piece. As a result, the two elements can be handled together and stored together.

The closure cap 9 preferably comprises a soft material; it preferably consists of this material. In particular, the material is preferably softer or more elastic than the material of the needle attachment piece 7. When the closure cap 9 is set onto the needle attachment piece 7, the closure cap 9 is preferably expanded elastically, so that it lies against the needle attachment piece 7, forming a seal. It preferably has a central projection 25 that comes into sealing engagement with an opening 27 of the needle attachment piece 7, in the set-on state, in order to further increase the sealing effect.

The soft or elastic material is preferably a thermoplastic elastomer (TPE). In another exemplary embodiment, it is possible that the material is a cross-linked thermoplastic elastomer (TPE-V). These materials are particularly suitable for primary contact with a medical and/or pharmaceutical formulation. The closure 3 and the entire syringe 1 are preferably configured in such a manner that the medical and/or pharmaceutical formulation exclusively comes into contact with materials that are suitable for primary contact. The needle attachment piece 7 preferably comprises glass, or preferably consists of glass. In another exemplary embodiment, a plastic, preferably a hard plastic is also possible as a material for the needle attachment piece 7. This piece is preferably configured in one piece with the syringe cylinder 5 and particularly preferably comprises the same material as the latter.

The securing cap 11 preferably comprises a comparatively hard material or preferably consists of it. In particular, the material is preferably harder than the material of the closure cap 9, so that the securing cap 11 can support the latter and absorb forces introduced into it. It also serves to protect the closure cap 9 from forces that act on it from the outside, so that it is not accidentally removed.

The hard material is preferably a thermoplastic polymer, particularly preferably polypropylene.

The securing ring 15, too, and preferably also the holding ring 13 comprise a comparatively hard material or consist of such a material. Particularly preferably, they comprise the same material as the securing cap 11, or consist of this material. The securing ring 15 and the holding ring 13 can fulfill their securing and holding function, respectively, particularly well if they consist of a comparatively hard material.

Preferably, however, the material of the holding ring 13 is at least slightly more elastic than the material comprised by the needle attachment piece 7. In this case, it is possible to push the holding ring 13 onto the needle attachment piece 7, where the ring can deform elastically because of the diameter of the needle attachment piece 7, which increases conically in FIG. 1. Below the undercut 17, it then springs back elastically, so that it firmly makes contact behind the undercut 17—viewed in the push-on direction—preferably under radial bias. For this purpose, the inside diameter of the holding ring 13 is coordinated, in suitable manner, with the outside diameter of the syringe 1 in the region in FIG. 1 underneath the undercut 17. It is then no longer possible to pull it off from the needle attachment piece 7 in destruction-free manner.

The securing ring 15 is preferably configured in such a manner, as the result of the selection of its wall thickness, that when it is pushed onto the holding ring 13 it can be widened slightly, where finally, the projection 19 springs in elastically behind the undercut 21—viewed in the push-on direction. The securing ring 15 then also lies against the holding ring 13, preferably under bias, so that it is no longer possible to pull it off again without destroying it.

In an exemplary embodiment that is not shown, the securing ring 15 and the holding ring 13 can be configured in one piece. They are then not joined together as described, but rather form a single, one-piece element that is set onto the needle attachment piece 7. They can particularly be produced as a one-piece element, using an injection-molding process. If they comprise the same material, a single injection-molding step is sufficient for this. They can, however, also comprise different materials, and then they can preferably be produced using a two-component injection-molding process.

The securing cap 11 and the closure cap 9 are also preferably produced as a one-piece element, using a two-component injection-molding process.

It is possible that the securing cap 11 and the securing ring 15 are produced as a one-piece element in a single injection-molding step, from the same material. In this connection, the at least one tear-off strip that connects the two pieces is also formed. The closure cap 9 is then preferably produced in a further injection-molding step.

The holding ring 13 can be produced separately. However, it is also possible to preferably produce it in the same injection-molding step, from the same material, together with the securing cap 11 and the securing ring 15.

In total, this shows that in a preferred exemplary embodiment, the entire closure 3 can be produced as a one-piece element, using a multi-component injection-molding process.

The closure now only has to be pushed onto the needle attachment piece 7 until the holding ring 13 engages behind the undercut 17—viewed in the push-on direction—in order to close off the syringe 1.

FIG. 2 shows a detail representation of the exemplary embodiment of the syringe 1 according to FIG. 1 in longitudinal section, in the closed state. Elements that are the same and have an equivalent function are provided with the same reference symbols, so that in this regard, reference is made to the above description. In FIG. 2, it is shown that the holding ring 15 in the present exemplary embodiment engages behind the undercut 21 with the projection 19, locking in place. Because the securing cap 11 is connected with the securing ring 15 by way of the at least one tear-off strip, it is indirectly attached to the needle attachment piece 7, because the securing ring 15 is attached to the holding ring 13, which in turn is attached to the needle attachment piece 7, in which it engages behind the undercut 17, essentially locking in place.

The needle attachment piece 7 and the closure cap 9 are shown more or less overlapping, because in FIG. 2, the deformation of the soft or elastic material of the closure cap 9 is not taken into consideration. From this representation, however, it is particularly clearly evident that in fact, the closure cap 9 is elastically deformed when it is set onto the needle attachment piece 7, so that the cap lies against the piece, forming a seal. In particular, the projection 25 ensures tight coverage of the opening 27.

In the following, the method for the production of a closure will be explained in greater detail:

First, either the securing cap 11 is injection-molded from a first, preferably hard material, or the closure cap 9 is injection-molded from a second, preferably soft material. Subsequently, either the closure cap 9 is injection-molded onto the securing cap 11 or the securing cap 11 is injection-molded onto the closure cap 9. In any case, the two elements are formed onto one another, in one piece, preferably using a two-component injection-molding process.

Preferably, the securing ring 15 is also formed in the same multi-component injection-molding process. In particular, it is possible to form it in the same injection-molding step together with the securing cap 11, if it comprises the same material as the latter. In this connection, the at least one tear-off strip is also formed at the same time, which strip connects the two elements with one another. The closure cap 9 is then formed onto the unit of securing cap 11 and securing ring 15, or this unit is formed onto the closure cap 9.

Preferably, the holding ring 13 is also formed in the same multi-component injection-molding process as the securing cap 11 and the closure cap 9.

In an embodiment of the method, it is possible to form the securing cap 11, the securing ring 15, and the holding ring 13 in one injection-molding step, from the same material. Subsequently, the closure cap 9 can then be formed onto this unit, or the unit can be formed onto the closure cap 9. In this way, the closure 3 is formed as a completely one-piece element that only has to be set onto the needle attachment piece 7 to close off the syringe 1.

In another embodiment of the method, it is possible to form the securing ring 15 and the holding ring 13 in the same multi-component injection-molding process as the securing cap 11 and the closure cap 9, but here, to provide at least one additional injection-molding step for the formation of the securing ring 15 and/or the holding ring 13. In particular, it is then possible that the securing ring 15 and/or the holding ring 13 comprise different materials from the securing cap 11 and/or from one another.

Particularly preferably, the securing cap, the securing ring and/or the holding ring is/are injection-molded from a material that comprises polypropylene, preferably consists of polypropylene.

The closure cap is preferably injection-molded from a material that comprises thermoplastic elastomer (TPE), preferably consists of it. In an embodiment of the method, it is possible to injection-mold the closure cap from a material that comprises cross-linked thermoplastic elastomer (TPE-V), preferably consists of it.

In a preferred embodiment of the method, the securing cap 11 and the closure cap 9 are locked into one another during injection molding. In this connection, at least one recess 29 is preferably provided on the closure cap 9, into which recess at least one projection 31 of the securing cap 11 engages. It is also possible to provide a recess on the securing cap 11, into which recess a corresponding projection of the closure cap 9 engages. Finally, it is possible to provide at least one recess and at least one projection, which engage into one another, locking into place, on both elements.

It is readily clear that the closure cap 9 and the securing cap 11 then comprise the corresponding engagement means, namely projections and/or recesses.

Preferably, the at least one recess 29 and the at least one projection 31 are configured in such a manner that axial forces can be introduced from the securing cap 11 into the closure cap 9. In this way, it is possible that the closure cap 9 is carried along when the securing cap 11 is separated and removed from the securing ring 15. In order to expose the needle attachment piece 7 for use of the syringe 1, it is then not necessary to first remove the securing cap 11 and then the closure cap 9, but rather both elements are removed at the same time when the closure cap 11 is removed.

Preferably, the at least one projection 31 and the at least one recess 29 are configured in such a manner that even forces that act in the circumference direction can be introduced from the securing cap 11 into the closure cap 9 or vice versa. In this way, relative rotation between the securing cap 11 and the closure cap 9 is effectively prevented. This increases the seal of the closure 3, because the closure cap 9 does not move in the securing cap 11, particularly cannot be rotated. Preferably, multiple recesses 29 and projections 31 are provided here—viewed in the circumference direction—particularly preferably at an equal angle distance from one another.

Because the securing cap 11 and the closure cap 9 are configured as a one-piece element, and particularly because the two elements are locked into one another, the construction height or expanse—viewed in the longitudinal and axial direction, respectively—of the closure 3 can be reduced. Specifically, if these elements are configured in two pieces, the securing cap 11 must not only surround the closure cap 9 but must also engage over it, in order to protect it from loss or unauthorized removal, or—viewed in the axial direction—to stabilize it. If, on the other hand, the elements are configured in one piece with one another, particularly locked into one another, it is sufficient if the securing cap 11 surrounds the closure cap 9—viewed in the circumference direction—as shown in FIG. 1 and in FIG. 2. Although the closure cap 9 is accessible from above in the representation of FIG. 2, it cannot be removed from the needle attachment piece 7 as long as the securing cap 11 is connected with the securing ring 15. The closure 3 can therefore—viewed in the longitudinal direction—have a shorter expanse or a smaller construction height.

With this, it is shown, in total, that the syringe, the closure and the method are particularly efficient and cost-saving, because production costs and storage costs as well as losses of small parts are effectively avoided. At the same time, the logistics effort in connection with the syringe 1 is clearly lower than in the case of known syringes.

1-16. (canceled)

17. A syringe comprising:
a syringe cylinder;
a needle attachment piece provided at a distal end of the syringe cylinders;
a closure including a closure cap that tightly seals the needle attachment piece; and
a securing cap surrounding the closure cap and attached to the needle attachment piece by way of a holding ring, the securing cap and the closure cap configured in one piece.

18. The syringe according to claim 17, further comprising a fastening means proximate the needle attachment piece for attachment of the holding ring.

19. The syringe according to claim 18, wherein the fastening means is selected from a group consisting of a groove, a bead and an undercut.

20. The syringe according to claim 17, further comprising a securing ring surrounding the holding ring, the securing cap removably attached to the securing ring.

21. A closure for a syringe, the closure comprising:
a closure cap for tightly sealing the syringe;
a securing cap surrounding the closure cap;
a holding ring for fastening the closure onto the syringe; and
a securing ring surrounding the holding ring, the securing cap removably attached to the securing ring;
wherein the closure cap and the securing cap are configured in one piece.

22. The closure according to claim 21, wherein at least one of the securing cap, the holding ring and the securing ring is constructed of a thermoplastic polymer.

23. The closure according to claim 22, wherein the thermoplastic polymer is polypropylene.

24. The closure according to claim 21, wherein the closure cap is constructed of a soft material.

25. The closure according to claim 24, wherein the soft material is TPE.

26. The closure according to claim 17, wherein the securing ring and the holding ring are configured in one piece.

27. The closure according to claim 17, wherein the securing cap and the closure cap are produced as a one-piece element, using a two-component injection-molding process.

28. The closure according to claim 17, wherein the securing ring and the holding ring are produced as a one-piece element, using an injection-molding process.

29. The closure according to claim 17, wherein the closure is produced as a one-piece element, using a multi-component injection-molding process.

30. A method for making the closure of claim 21, the method comprising:
injection-molding the securing cap of a first material; and
injection-molding of a closure cap of a second material by injection-molding the closure cap onto the securing cap, or injection-molding the securing cap onto the closure cap;
wherein the first material is a relatively hard material and the second material is a relatively soft material.

31. The method according to claim 30, wherein the securing ring and the holding ring are formed in a same multi-component injection-molding process as the securing cap and the closure cap.

32. The method according to claim 30, wherein the securing cap, the securing ring, and the holding ring are formed in one step, from a same material.

33. The method according to claim 30, wherein at least one of the securing cap, the securing ring and the holding ring is injection-molded from polypropylene.

34. The method according to claim 30, wherein the closure cap is injection-molded from TPE.

35. The method according to claim 30, wherein the securing cap and the closure cap are locked into one another during injection-molding.
